(19) United States
(12) Patent Application Publication
(10) Pub. No.: US 2014/0114241 A1
(43) Pub. Date: Apr. 24, 2014

(54) COATING OF SURFACES FOR SUSTAINED DRUG RELEASE

(71) Applicant: CAPSULUTION PHARMA AG, Berlin (DE)

(72) Inventors: Andreas Voigt, Berlin (DE); Lutz Kröhne, Berlin (DE)

(73) Assignee: CAPSULUTION PHARMA AG, Berlin (DE)

(21) Appl. No.: 13/978,139

(22) PCT Filed: Jun. 1, 2011
(86) PCT No.: PCT/EP11/59063

§ 371 Date: Oct. 4, 2013

(30) Foreign Application Priority Data
Jun. 3, 2010 (EP) 10164857.4

(51) Int. Cl. A61L 31/10; A61L 27/54; A61L 27/34; A61L 31/16

(52) U.S. Cl. CPC A61L 31/10; A61L 31/16; A61L 27/54; A61L 27/34
USPC 604/93.01; 427/2.21

(57) ABSTRACT

The present invention relates to a method for coating a surface of a substrate with a drug for sustained release: (i) providing a substrate with a surface to be coated, (ii) depositing at least one bilayer on at least a portion of said surface, wherein one layer of the bilayer comprises a polyelectrolyte and the other layer comprises a pharmaceutically active ingredient, and wherein the two layers of the bilayer are oppositely charged under conditions of the deposition and one layer of the bilayer has a substantially different net charge under physiological conditions. The invention also pertains to substrates with microscopic or macroscopic surfaces coated with the method according to the invention.

### TECHNICAL FIELD OF THE INVENTION

The present invention is in the field of medicinal products. Particularly, the present invention relates to the coating of surfaces of microscopic and macroscopic substrates with drugs for sustained drug release.

### BACKGROUND OF THE INVENTION

Drug delivery technologies are formulation technologies that modify drug release profile, absorption, distribution and elimination for the benefit of improving product efficacy and safety, as well as patient convenience and compliance. For many medical applications and pharmaceutically active agents, a controlled drug delivery and release is desired. While some applications require a rapid release of a drug at the desired target, other applications require a delayed or sustained drug release. This does not only apply for the administration of drug s for therapeutic, preventive or diagnostic purposes but also to certain implantable or insertable medical devices such as catheters or stents.

Numerous approaches exist for targeted delivery and/or sustained drug formulations. For example, sustained release formulations include liposomes, drug loaded biodegradable microspheres and drug polymer conjugates.

### SUMMARY OF THE INVENTION

The present invention provides a method for coating surfaces of microscopic or macroscopic substrates with a drug for sustained release of the drug.

The present invention is in part based on the inventors' finding that layer-by-layer coatings of surfaces of microscopic or macroscopic substrates have particularly advantageous properties when layers of the pharmaceutically active ingredient (the “drug”) and polyelectrolyte layers are applied in an alternating manner, wherein under the conditions of coating the polyelectrolyte layers and the drug layers have opposite net charges and under release conditions, e.g. physiological conditions, the two layers have substantially the same net charge. Hence either the polyelectrolyte or the drug is an amphoteric substance that substantially changes its net charges under the conditions were the release of the drug is desired, e.g. at physiological conditions.

The invention relates in particular to a method for coating a surface of a substrate with a drug for sustained release:
(i) providing a substrate with a surface to be coated,
(ii) depositing at least one bilayer on at least a portion of said surface,
   wherein one layer of the bilayer comprises a polyelectrolyte and the other layer comprises a pharmaceutically active ingredient, and
   wherein the two layers of the bilayer are oppositely charged under conditions of the deposition and one layer of the bilayer has a substantially different net charge under physiological conditions.

The present invention also relates to a substrate comprising
(i) a surface, and
(ii) a multilayer coating at least on a portion of said surface,
   wherein the multilayer comprises one or more bilayers having two layers of opposite charge at deposition and storage conditions, and
   wherein one layer of the bilayer comprises a polyelectrolyte and the other layer comprises a pharmaceutically active ingredient, and
   wherein one layer of the bilayer substantially changes its net charge when subjected to physiological conditions.

### DESCRIPTION OF DRAWINGS

FIG. 1 shows a schematic representation of the coating of a surface of a substrate according to a particular embodiment of the invention. 11: surface to be coated; 21 basic layer (optional); 31: multilayer comprising bilayers; 33: bilayer comprising two oppositely charged layers; 35: layer of polyelectrolyte; 36: layer comprising pharmaceutically active ingredient; 41: top layer (optional).

FIG. 2 shows the QCM (Quartz Crystal Microbalance) frequency shift vs layer number for Hya/BMP-2 multilayers constructed at pH 4 (see Example 1).

FIG. 3 shows the release of BMP-2 in PBS (phosphate buffered saline) at pH 7.4, storage was at 37° C. (see Example 1).

FIG. 4 illustrates layer build-up and shows the decrease in frequency (left axis) and increase in adsorbed mass (right axis), respectively, per layer for a crystals coated with PEI-[ChonS/GelB]₁₂ at pH 2.5 (Example 3).

FIG. 5 illustrates pH-dependent desorption of polyelectrolyte layers in mass vs. time at pH 7.4 for crystals coated with PEI-[ChonS/GelB]₁₂ at pH 2.5 (Example 3).

FIG. 6 illustrates layer build-up and shows the decrease in frequency (left axis) and increase in adsorbed mass (right axis), respectively, per layer for a crystals coated with PEI-[ChonS/HSA]₁₂ at pH 2.5 (Example 4).

FIG. 7 illustrates pH-dependent desorption of polyelectrolyte layers in mass vs. time at pH 7.4 for crystals coated with PEI-[ChonS/HSA]₁₂ at pH 2.5 (Example 4).

FIG. 8 illustrates the net charge of the coated particles in terms of zeta potential during coating. Shown is the zeta potential of PEI-(ChonS/MP)₅ LBL-coating onto CaCO₃ particles (Precarb 720) at pH 3.5 to control layer build-up (Example 5).

FIG. 9 illustrates the increase of the overall concentration of MP in the multilayer coating after 2, 4, 6, 8 and 10 layers of coating. Shown is the increase in MP drug concentration during LBL-coating (PEI-(ChonS/MP)₅) onto CaCO₃ particles (Precarb 720) at pH 3.5; measurement of supernatants after coating with MP layer by UV-Vis spectroscopy; 2-fold experiment (Example 5)

FIG. 10 illustrates the release of the MP at physiological pH. Shown is the release of MP at pH 7.4 (PBS) out of CaCO₃ particles (Precarb 720) coated with LBL-coating PEI-(ChonS/MP)₅ at pH 3.5; measured by UV-Vis spectroscopy; 2-fold experiment (Example 5)

### DETAILED DESCRIPTION OF THE INVENTION

In a first aspect the present relates to a method for coating a surface of a substrate with a drug for sustained release:
(i) providing a substrate with a surface to be coated,
(ii) depositing at least one bilayer on at least a portion of said surface,
   wherein one layer of the bilayer comprises a polyelectrolyte and the other is layer comprises a pharmaceutically active ingredient, and
   wherein the two layers of the bilayer are oppositely charged under conditions of the deposition and one layer of the bilayer has a substantially different net charge under physiological conditions.

It is particularly preferred that the two layers of the bilayer have the same net charge under physiological conditions. Preferably, herein, the depositing of the at least one bilayer on the surface takes place at a pH<6 or a pH>8, preferably at a pH<5.4 or at a pH>9.

In one embodiment of the invention the pharmaceutically active ingredient is an amphoteric substance that has an isoelectric point IEP>9.4 or IEP<5.4. In another embodiment of the invention the polyelectrolyte is an amphoteric substance that has an isoelectric point IEP>9.4 or IEP<5.4.

The present invention can be applied to surfaces of microscopic substrates as well as to surfaces of macroscopic substrates. Microscopic substrates in the context of the present invention include solid particles, porous particles, particularly magnetic beads, nanoparticles and microparticles.

The particles of the present invention may be porous or non-porous. They may be of organic or inorganic material. Organic particles and beads may for instance be particles or beads of gelatin, chitosan, albumin, alginate, poly(lactic-co-glycolic acid) (PLGA), polyglycolic acid (PGA), or polylactic acid (PLA). Inorganic particles or beads may for example be particles of calcium carbonate (CaCO₃), silica (SiO₂), gold, silver, magnetite, maghemite.

Macroscopic substrates include for example implantable or insertable medical devices. Implantable or insertable medical devices benefiting from the present invention include any medical device for which controlled release of a therapeutic agent is desired. Examples of such medical devices include for instance, catheters (e.g., renal or vascular catheters such as balloon catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, orthopedic implants, temporary implants in the mouth (e.g., temporarily crown jackets which release a pain killer), artificial implanted lenses and biopsy devices. Hence, the drug-eluting implantable or insertable medical device of the invention may for example be an expandable device and/or comprises an inflatable portion. The device may also a be removable device. Preferred examples of implantable or insertable medical device according to the invention include stents, catheters, particularly balloon catheters, pacemakers, or artificial vessels (permanent or transient) or parts of such devices such as balloons in the case of balloon catheters. The medical devices of the present invention include medical devices that are used for either systemic treatment or for the localized treatment of any mammalian tissue or organ. Examples include tumors; organs including the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; dermal tissue; cartilage; and bone.

Surfaces of the implantable or insertable medical devices may for example be plastic, metal glass or ceramic surfaces. The surface can e.g. be polyamide-based. In the case of balloons, the surface for example is based on nylon-12, nylon-11 or nylon-6 or nylon-co-polymers, such as Pebax. Surfaces of balloons may also be made out of polyurethane or PET.

However, also surfaces of other macroscopic substrates may be temporarily or permanently coated with a drug according to the present invention. Such substrates may for example be surfaces of gold, silver, metal and glass.

Preferably herein, the substrate is a microsphere, porous particle, magnetic bead, a nanoparticle, a microparticle, or an implantable or insertable medical device.

The terms “therapeutic agent”, “drug”, “pharmaceutically active agent” and “pharmaceutically active ingredient” and other related terms may be used interchangeably herein.

As used herein, “treatment” refers to the prevention of a disease or condition, the reduction or elimination of symptoms associated with a disease or condition, or the substantial or complete elimination a disease or condition. Preferred subjects are mammalian subjects and more preferably human subjects.

Multilayers can be assembled using various known layer-by-layer techniques. Layer-by-layer techniques involve coating various substrates using charged materials, e.g. polyelectrolytes, via electrostatic, self-assembly. In the layer-by-layer technique, a first layer having a first net charge is typically deposited on an underlying substrate, followed by a second layer having a second net charge that is opposite in sign to the net charge of the first layer, and so forth. The two consecutive oppositely charged layers are together designated as bilayer. The charge on the outer layer is reversed upon deposition of each sequential layer or at least the net charge is substantially reduced. To the extent that the surface to be coated does not have an inherent net surface charge, a surface charge may be provided. For example, where the surface to be coated is conductive, the surface charge can be provided by applying an electrical potential to the same. Once a first layer is established in this fashion, a polyelectrolyte layer having a second net charge that is opposite in sign to the net charge of the first layer can readily be applied, and so forth. As another example, a surface charge can be provided by exposing the surface to be coated to a charged amphiphilic substance. Amphiphilic substances include any substance having hydrophilic and hydrophobic groups. Where used, the amphiphilic substance should have at least one electrically charged group to provide the substrate surface with a net electrical charge. Therefore, the amphiphilic substance that is used herein can also be referred to as an ionic amphiphilic substance. Amphiphilic polyelectrolytes can be used as ionic amphiphilic substances. For example, a polyelectrolyte comprising charged groups (which are hydrophilic) as well as hydrophobic groups, such as polyethylenimine (PEI) or poly(styrene sulfonate) (PSS), can be employed. Cationic and anionic surfactants can also be used as amphiphilic substances. Cationic surfactants include quaternary ammonium salts (R4N+X″), for example, didodecyldimethylammonium bromide (DDDAB), alkyltrimethylammonium bromides such as hexadecyltrimethylammonium bromide (HDTAB), dodecyltrimethylammonium bromide (DTMAB), myristyltrimethylammonium bromide (MTMAB), or palmityl trimethylammonium bromide, or N-alkylpyridinium salts, or tertiary amines (R3NH+X″), for example, cholesterol-3,3-N-(dimethyl-aminoethyl)-carbamate or mixtures thereof, wherein X″ is a counter-anion, e.g. a halogenide. Anionic surfactants include alkyl or olefin sulfate (R—OSO3M), for example, a dodecyl sulfate such as sodium dodecyl sulfate (SDS), a lauryl sulfate such as sodium lauryl sulfate (SLS), or an alkyl or olefin sulfonate (R—SO3M), for example, sodium-n-dodecyl-benzene sulfonate, or fatty acids (R—COOM), for example, dodecanoic acid sodium salt, or phosphoric acids or cholic acids or fluoro-organics, for example, lithium-3-[2-(perfluoroalkyl)ethylthio]propionate or mixtures thereof, where R is an organic radical and M is a counter-cation. Hence, the method may in particular embodiments comprise the step of depositing a layer of an amphiphilic substance to said portion of the surface before depositing the multilayer structure. Such a layer is herein designated “basic layer” (21).

In other embodiments, a surface charge is provided by adsorbing cations (e.g., protamine sulfate, polyallylamine, polydiallyldimethylammonium species, polyethyleneimine, chitosan, gelatin, spermidine, among others) or anions (e.g., polyacrylic acid, sodium alginate, polystyrene sulfonate, Eudragit S, gelatin (gelatin is an amphoteric polymer, hence it fits in both categories depending how it is being prepared), hyaluronic acid, carrageenan, chondroitin sulfate, carboxymethylcellulose, among others) to the surface to be coated as a first charged layer. Although full coverage may not be obtained for the first layer, once several layers have been deposited, a full coverage should ultimately be obtained, and the influence of the substrate is expected to be negligible. The species for establishing surface charge can be applied to region of the surface to be coated by a variety of techniques. These techniques include, for example, spraying techniques, dipping techniques, rinsing techniques, roll and brush coating techniques, techniques involving coating via mechanical suspension such as air suspension, ink jet techniques, spin coating techniques, web coating techniques and combinations of these processes. Alternatively or additionally, an activation of the surface can be performed, for instance by chemical etching with e.g. a H₂O/HCl/H₂O₂ mixture and/or a H₂O/NH₃/H₂O₂ mixture. This results in temporary charges on the surface which in turn promote the adsorption of polyelectrolytes to the surface. An exemplary protocol for chemical etching is given in Example 2.

Once a sufficiently charged substrate is obtained, it can be coated with a layer of an oppositely charged substance, preferably a polyelectrolyte. Multilayers are formed by repeated treatment with alternating oppositely charged substances, i.e., by alternating treatment with cationic and anionic substances. The polymer layers self-assemble by means of electrostatic layer-by-layer deposition, thus forming a multilayered coating over the surface to be coated. In accordance with the invention one of the layers comprises a polyelectrolyte and the other layer comprises the pharmaceutically active ingredient.

Polyelectrolytes are polymers having charged (e.g., ionically dissociable) groups. Usually, the number of these groups in the polyelectrolytes is so large that the polymers in dissociated form (also called polyions) are water-soluble. Depending on the type of dissociable groups, polyelectrolytes are typically classified as polyacids and polybases. When dissociated, polyacids form polyanions, with protons being split off. Polyacids include inorganic, organic and bio-polymers. Examples of polyacids are polyvinylphosphoric acids, polyvinylsulfonic acids, polyvinylsulfonic acids, polyvinylphosphonic acids and polyacrylic acids. Examples of the corresponding salts, which are also called polysalts, are polyvinylphosphates, polyvinylsulfates, polyvinylsulfonates, polyvinylphosphonates and polyacrylates. Polybases contain groups which are capable of accepting protons, e.g., by reaction with acids, with a salt being formed. Examples of polybases having dissociable groups within their backbone and/or side groups are polyallylamine, polyethylimine, polyvinylamine and polyvinylpyridine. By accepting protons, polybases form polycations. Quaternary ammonium groups are also preferred cationic groups in the context of the present invention. For example polydiallyl dimethyl ammonium chloride (PDADMAC) is a very strong cationic charged polyelectrolyte.

Suitable polyelectrolytes according to the invention include those based on biopolymers, for example, alginic acid, gummi arabicum, nucleic acids, pectins and proteins, chemically modified biopolymers such as carboxymethyl cellulose and lignin sulfonates, and synthetic polymers such as polymethacrylic acid, polyvinylsulfonic acid, polyvinylphosphonic acid and polyethylenimine. Linear or branched polyelectrolytes can be used. Using branched polyelectrolytes can lead to less compact polyelectrolyte multilayers having a higher degree of wall porosity. Polyelectrolyte molecules can be crosslinked within or/and between the individual layers, e.g. by crosslinking amino groups with aldehydes, for example, to increase stability. However, it is preferred in the context of the present invention that the polyelectrolytes are not cross-linked. Furthermore, amphophilic polyelectrolytes, e.g. amphiphilic block or random copolymers having partial polyelectrolyte character, can be used to affect permeability towards polar small molecules. Such amphiphilic copolymers consist of units having different functionality, e.g. acidic or basic units, on the one hand, and hydrophobic units, on the other hand (e.g., polystyrenes, polydienes or polysiloxanes), which can be present in the polymer as blocks or distributed statistically. Suitable polyelectrolytes include low-molecular weight polyelectrolytes (e.g., polyelectrolytes having molecular weights of a few hundred Daltons) up to macromolecular polyelectrolytes (e.g., polyelectrolytes of biological origin, which commonly have molecular weights of several million Daltons). Preferably herein, at least one of the used polyelectrolytes has a molecular weight of below 100 kDa, preferably below 10 kDa. Specific examples of polycations include protamine sulfate polycations, poly(allylamine) polycations (e.g., poly(allylamine hydrochloride) (PAH)), polydiallyldimethylammonium (PDADMAC) polycations, polyethyleneimine (PEI) polycations, chitosan polycations, spermidine polycations and albumin polycations. Specific examples of polyanions include poly(styrenesulfonate) polyanions (e.g., poly(sodium styrene sulfonate) (PSS)), polyacrylic acid polyanions, sodium alginate polyanions, hyaluronic acid polyanions, carrageenan polyanions, chondroitin sulfate polyanions, carboxymethylcellulose polyanions and albumin polyanions.

By using polyelectrolytes that are biodisintegrable, the release of the therapeutic agent can be further controlled based on the rate of disintegration of the polyelectrolyte layers. Moreover, as indicated above, implantable or insertable medical articles containing a biodisintegrable multilayer polyelectrolyte coating leave behind only the underlying ceramic or metallic structure once the therapeutic agent is released from the medical article. As used herein, a “biodisintegrable” material is a material which undergoes dissolution, degradation, resorption and/or other disintegration processes upon administration to a patient. Preferred examples of biodisintegrable polyelectrolytes include protamine sulfate, gelatin, spermidine, albumin, carrageenan, chondroitin sulfate, heparin, other polypeptides and proteins, and DNA, among others. As with species for establishing surface charge (described above), the polyelectrolyte layers can be applied to the surface to be coated by a variety of techniques including, for example, spraying techniques, dipping techniques, rinsing techniques, roll and brush coating techniques, techniques involving coating via mechanical suspension such as air suspension, inkjet techniques, spin coating techniques, web coating techniques and combinations of these processes. Preferably, herein, the polyelectrolyte layers are applied by spraying, brushing or by immersion of the surface to be coated into a solution comprising the respective polyelectrolyte.

Tables 1 to 3 list preferred cationic, anionic and amphoteric polyelectrolytes, respectively.

TABLE 1
Examples of cationic polyelectrolytes

| Polymers | molecular weight (MW) | |
|---|---|---|
| Protamine (Prot) | ca. 4 800 | Da |
| Chitosan (Chi) | wide range | |
| Polyethylenimine (PEI) | 10−>300 | kDa |
| Poly-L-arginine (PLArg) | 10−>300 | kDa |
| Poly-L-lysine (PLL) | 10−>300 | kDa |
| Spermine | 238-348 | Da |
| Spermidine | 145-255 | Da |

TABLE 2
Examples of anionic polyelectrolytes

| Polymers | MW | |
|---|---|---|
| Carboxymethylcellulose | wide range | |
| Hyaluronic acid | 1.6-3.3 | MDa |
| Chondroitinsulfate | 15-50 | kDa |
| Heparin | 3-30 | kDa |

| | |
|---|---|
| Alginate acid | wide range |
| Carrageenan | ι, κ, λ |
| Gums (Xynthan, Acacia . . .) | wide range |

TABLE 3
Examples of amphoteric polyelectrolytes

| Substance | MW | Behavior |
|---|---|---|
| Serum Albumin | 66 kDa | positive < IEP 4.7 > negative |
| Gelatin A | wide range | positive < IEP 8-9 > negative |
| Gelatin B | wide range | positive < IEP 4.8-5.4 > negative |
| Collagen | 130 kDa | positive in neutral/acid solution |
| | | positive and negative matrices |

Also the layers comprising the pharmaceutically active ingredient can be applied to the surface to be coated by a variety of techniques including, for example, spraying techniques, dipping techniques, rinsing techniques, roll and brush coating techniques, techniques involving coating via mechanical suspension such as air suspension, inkjet techniques, spin coating techniques, web coating techniques and combinations of these processes. Preferably, herein, the layers comprising the pharmaceutically active ingredient are applied by spraying, brushing or by immersion of the surface to be coated into a solution comprising the comprising the pharmaceutically active ingredient.

Preferably, herein, a plurality of bilayers is deposited on at least the portion of the surface to form a multilayer of layers with alternating charges. According to the method of the invention, preferably between 2 and 1000, more preferably between 2 and 250 layers are applied to the surfaces and a multilayer of alternating charge is formed. Hence, the coating of the microscopic or macroscopic surfaces according to the invention preferably comprises a multilayer having between 1 and 500, preferably between 1 and 125 bilayers.

In some preferred embodiments the coating has an overall thickness of from 2 nm to 500 nm, preferably 100 nm to 50 nm, more preferably 0.4 nm to 10 nm.

As described herein above, the polyelectrolytes can, inter alia, be synthetic polymers, biopolymers such as polypeptides, proteins, polysaccharides, oligosaccharides, nucleic acids and derivates of biopolymers such as chemically modified biological polymers.

The net charge of a polyelectrolyte or the pharmaceutically active ingredient may depend on the pH of the surrounding solution. For example some polyelectrolytes or pharmaceutically active ingredients may be amphoteric. An amphoteric substance is a substance that can react as either an acid or base. Amphoteric substances have an isoelectric point (pI or IEP), i.e. a pH at which they have no net charge and are thus neutral. Above the IEP the amphoteric substance is deprotonated and thus has a negative net charge. Below the IEP the amphoteric substance is protonated and thus has a positive net charge. Hence, whether a given amphoteric substance is a polyanion or a polycation depends on the surrounding pH. The present inventors have in a specific embodiment exploited this fact by using an amphoteric layer that has a different net charge during coating, i.e. oppositely charged than the neighbouring layers, than under in situ conditions or release conditions, e.g. under physiological conditions. When a substrate with a coating comprising such an amphoteric substance is brought to physiological pH, i.e. by administering, implanting or inserting the coated microscopic or macroscopic substrate to a subject, the net charge of the amphoteric substance changes, is resulting in a disintegration of the multilayer. This, in turn, leads to the release of the pharmaceutically active ingredient to the surrounding (target) tissue. Properties of the sustained release such as release rate, lack time, overall time of release etc. can for example be controlled by the selection of the polyelectrolyte and/or the numbers of layers applied to the surface.

“Physiological conditions”, particularly “physiological pH”, herein refers to the conditions, particularly the pH, at the place to which the coated substrate is administered, delivered, targeted, implanted or inserted, i.e. the conditions, particularly the pH, in situ or in vivo. In the case of blood vessels, for example, the pH is typically between 7.35 and 7.45, preferably around 7.4. The osmolarity in whole blood plasma is typically between 250 and 330 mosmol/kg, mostly between 275 and 299 mosmol/kg.

The pharmaceutically active ingredient in the context of the present invention is preferably water-soluble. It is preferably a water soluble drug according to groups I or III of the Biopharmaceutics Classification System (BCS) (FDA). In some embodiments the pharmaceutically active ingredient is an amphoteric substance that has a substantially different net charge at deposition conditions than at the conditions were the release of the pharmaceutically active ingredient is desired.

The pharmaceutically active ingredient in the context of the present invention is for instance substance for inhibiting cell proliferation or inflammatory processes, an anti-cancer drug, an antibiotic, a growth factor, a hormone, an antibody or functional fragment thereof, a cytostatic, an immunosuppressant or an antioxidant. Preferred pharmaceutically active ingredients in the context of the present invention are anti-TNF alpha antibodies and hormones and inhibitors of growth factors.

Table 4 lists some very particular proteins that are pharmaceutically active ingredients together with their isoelectric point and molecular weight.

TABLE 4
Examples of pharmaceutically active ingredients with
their isoelectric point (IEP) and molecular weight

| Pharmaceutically active ingredient | IEP | Molecular weight [kDa] |
|---|---|---|
| Bone morphogenetic protein 2 (BMP-2) | 8.2 | 26 |
| Bone morphogenetic protein 7 (BMP-7) | 7.7 | 15 |
| Lysozyme | 9-11 | 14.3 |
| IGF-I | 8.6 | 7.5 |
| VEGF | 8.6 | 46 |
| TGF-β1 | 9.5 | 25 |

According to the invention, the coating may additionally also comprise a layer comprising one or more substances that facilitate dissolution of the multilayer upon insertion or implantation of the device. Such substances include for example enzymes that cleave polyelectrolytes, e.g. polysaccharides. Such enzymes are for example chitinase, esterase, peptidase, and lysozyme.

The layer comprising the pharmaceutically active ingredient may in a particular embodiment further comprises a polyelectrolyte, an organic or inorganic salt, or organic or inorganic particles with low water solubility.

The present invention also relates to a substrate obtained or obtainable by a method according to the methods of the invention.

The invention, hence, in one aspect pertains to a substrate comprising
(i) a surface, and
(ii) a multilayer coating at least on a portion of said surface,
   wherein the multilayer comprises one or more bilayers having two layers of opposite charge at deposition and storage conditions, and
   wherein one layer of the bilayer comprises a polyelectrolyte and the other layer comprises a pharmaceutically active ingredient, and
   wherein one layer of the bilayer substantially changes its net charge when subjected to physiological conditions.

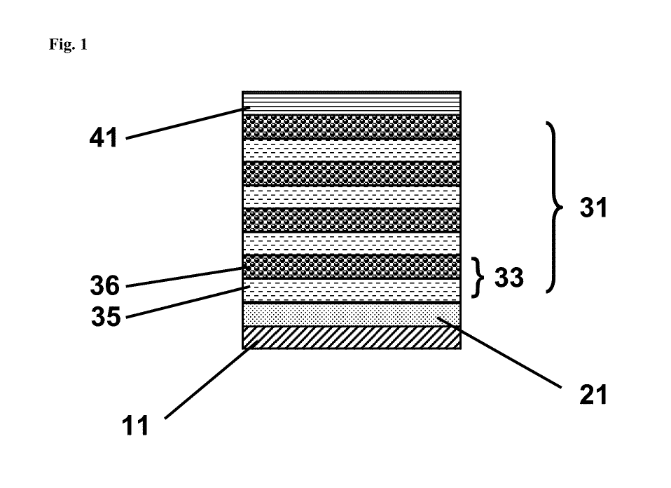

One particular embodiment of the coated substrate according to the invention is illustrated in FIG. 1.

The coated microscopic and macroscopic substrates according to the invention, e.g. the particles, porous particles or implantable or insertable medical devices, may be used in a wide spectrum of medical, veterinary, pharmaceutical and diagnostic applications. For example, the coated microscopic and macroscopic substrates according to the invention may be used in the treatment of tumours, for creating open passages in the body, for the treatment of vascular diseases or circulatory disturbances, for facilitating wound healing or the healing of bone fractures, for the treatment of ophthalmic diseases, in surgery, for the treatment of gynecological diseases and conditions, for the treatment of articular and bone diseases, for the treatment of stenosis or in the prophylaxis of restenosis.

Particularly, implantable or insertable medical devices coated according to the invention may be used in the treatment of tumours, for creating open passages in the body, for the treatment of vascular diseases or circulatory disturbances, for facilitating wound healing or the healing of bone fractures, for the treatment of ophthalmic diseases, in surgery, for the treatment of gynecological diseases and conditions, for the treatment of articular and bone diseases, for the treatment of stenosis or in the prophylaxis of restenosis.

Hence, the invention in one aspect also relates to the use of an implantable or insertable medical device according to the invention to provide a means for the treatment of tumours, for creating open passages in the body, for the treatment of vascular diseases or circulatory disturbances, for facilitating wound healing or the healing of bone fractures, for the treatment of ophthalmic diseases, for the use in surgery, for the treatment of gynecological diseases and conditions, for the treatment of articular and bone diseases, for the treatment of stenosis or for the prophylaxis of restenosis.

Microscopic substrates, particularly the particles according to the invention, e.g. microspheres, porous particles, magnetic beads, nanoparticles or microparticles, coated with a drug in accordance with the invention may for example be used in the treatment of diseases, e.g. tumours, vascular diseases or circulatory disturbances, ophthalmic diseases, gynecological diseases and conditions, articular and bone diseases, for facilitating wound healing or the healing of bone fractures.

The invention therefore relates in a particular aspect to the use of microscopic substrates, particularly the particles according to the invention, e.g. microspheres, porous particles, magnetic beads, nanoparticles or microparticles, coated with a drug in accordance with the invention for the manufacture of a medicament for the treatment of diseases, e.g. tumours, vascular diseases or is circulatory disturbances, ophthalmic diseases, gynecological diseases and conditions, articular and bone diseases, for facilitating wound healing or the healing of bone fractures.

The particles according to the invention may for instance be used for topical (i.e. local) or systemic (i.e. enteral or parenteral) administration. They may for example be used in oral, nasal, rectal, intramuscular, intradermal, intravenous, subcutaneous or other administration routes.

The following examples illustrate particular embodiments and aspects of the present invention. However, they are not limiting the scope of the invention.

### EXAMPLES

### Example 1

### Coating of a Part of a Titanium Hip Prosthesis with BMP 2—Hyaluronic Acid Bilayers at pH 4

Polymer solution 1: containing 0.2 mg/ml BMP-2, pH 4 (cationic charged).
Polymer solution 2: containing 0.2 mg/ml Hyaluronic acid (Hya), pH 4 (anionic charged)
Washing: water pH 4 (adjusted with HCl/NaOH)

### Purification and Pre-Treatment of Prostheses:

to be coated parts of the prostheses have been placed in a glass staining trough containing a mixture of 143 ml deionised water, 28.6 ml hydrogen peroxide and 28.6 ml ammonium hydroxide;
the glass staining trough was placed in a water bath and heated;
after reaching a temperature of 60° C. the through remained in the water bath for another 30 min at this temperature;
subsequently prostheses were washed three times with deionised water (3 glass beakers were filled with deionised water

### LbL Coating:

the coating was performed in glass staining troughs
the troughs were each filled with 200 ml of the respective polymer solution (Polymer solution 1 and 2)
subsequently the prostheses were immerged for 1 min into the respective trough for the coating with the first layer;
subsequently the prostheses were washed three times in water pH 4 (3 troughs of water);
then the prostheses were transferred to the next trough comprising the next polymer solution (1 min coating)
the washing and coating steps were repeated as required and according to the desired sequence of layers; each coating step lasted 1 min;
for storage the prostheses were dried.

### Sequence of Coating:

### (BMP-2/Hya) 5—BMP-2

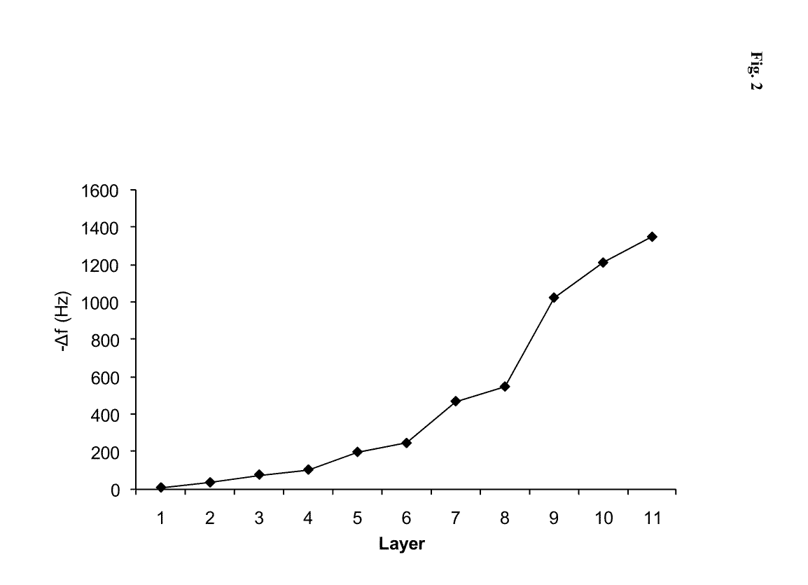

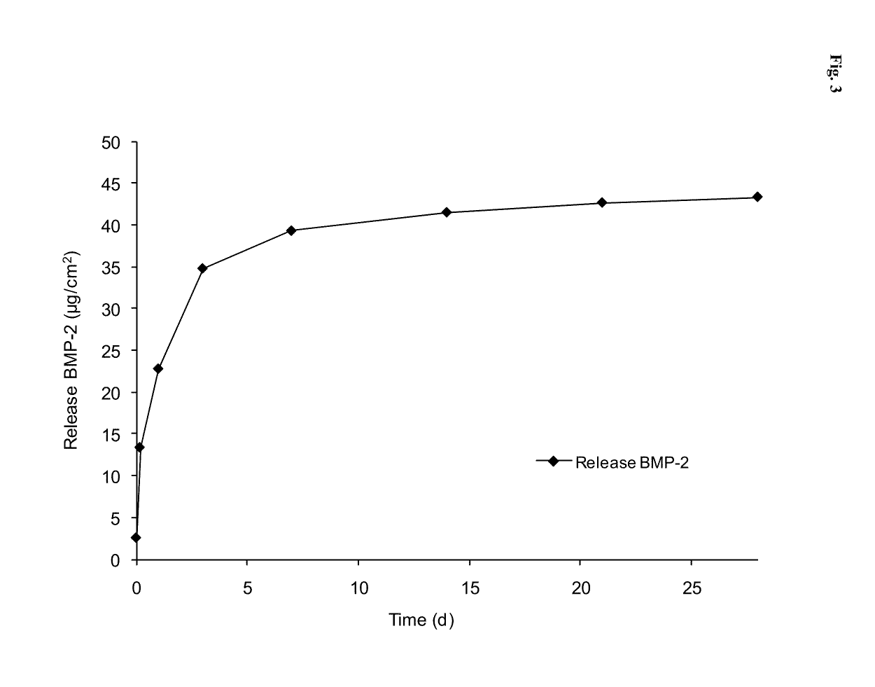

FIG. 2 shows the build-up at pH 4. FIG. 3 shows the release at physiological pH.

### Example 2

### Chemical Etching of the Surface to be Coated

The surface of e.g. a medical device can be prepared for coating with multilayers by chemical etching. The following protocol is based on purification protocols of silicon wafers as established by the Radio Corporation of America (RCA) (W. Kern, Cleaning Solutions Based on Hydrogen Peroxide for Use in Silicon Semiconductor Technology RCA Review 31, 187-206, 1970).

The so-called RCA purification id based on a two-step oxidizing and complexing treatment of the surface with hydrogen peroxide solutions. In the first step, an alkaline mixture with high pH and in the second step an acidic mixture with low pH is used.

In the first step a mixture of concentrated ammonium hydroxide (29%), concentrated hydrogen peroxide (30%) and water is used, typically in a ratio of 1:1:5 (NH₃:H₂O₂:H₂O, % (v/v)).

In the second step a mixture of concentrated hydrochloric acid (37%), concentrated hydrogen peroxide (30%) and water is used, typically in a ratio of 1:1:6 (HCl:H₂O₂:H₂O, % (v/v)).

Both steps are performed at elevated temperature, e.g. 60-80° C. for about 10 min.

For the coating, it is not required to apply both steps. Generally, the alkaline step is sufficient.

### Example 3

### Build-Up of Chondroitin Sulphate/Gelatine B Polyelectrolyte Layers and Desorption vs. pH

Example 3 illustrates an embodiment in which a surface (here: surface of crystals) are coated with polyelectrolyte multilayer layer (LbL: layer-by-layer). The layer is subsequently released at a physiological pH.

### Materials: Layer Build-Up

PEI-[ChonS/GelB]12 at pH 2.5
PE solution 1: Polyethylenimmine (PEI) (0.1 g/l) pH 2.5 containing 0.154 M NaCl
PE solution 2: Chondroitin sulphate (ChonS) (0.1 g/l) pH 2.5 containing 0.154 M NaCl
PE solution 3: Gelatine B (GelB) (0.1 g/l) pH 2.5 containing 0.154 M NaCl
Washing water: MilliQ-Water adjusted to pH 2.5

### Materials: Desorption of Layers at pH 7.4

Phosphate buffer (PBS) with Tween20 at pH 7.4

### Equipment:

Quartz Crystal Micro Balance (QCM-D)

### Pre-Treatment of Crystals:

The crystals to be coated were treated with polymer solution 1 (basic layer)

### LbL Coating:

after pre-treatment the crystals were coated with polymer solution 2 (anionic charge) at pH 2.5 for 2 min followed by a washing sequence with water at pH 2.5
subsequently the crystals were rinsed with polymer solution 3 (cationic charge) at pH 2.5 for 2 min followed by a washing sequence with water at pH 2.5
the washing and coating steps were repeated as required and according to the desired sequence of layers

### Sequence of Coating:

PEI-[ChonS/GelB]₁₂

### Desorption of PE Layers at pH 7.4:

rinse of crystals with PBS pH 7.4 solution for 1 min
residence time=29 min
total investigation time=16 hrs

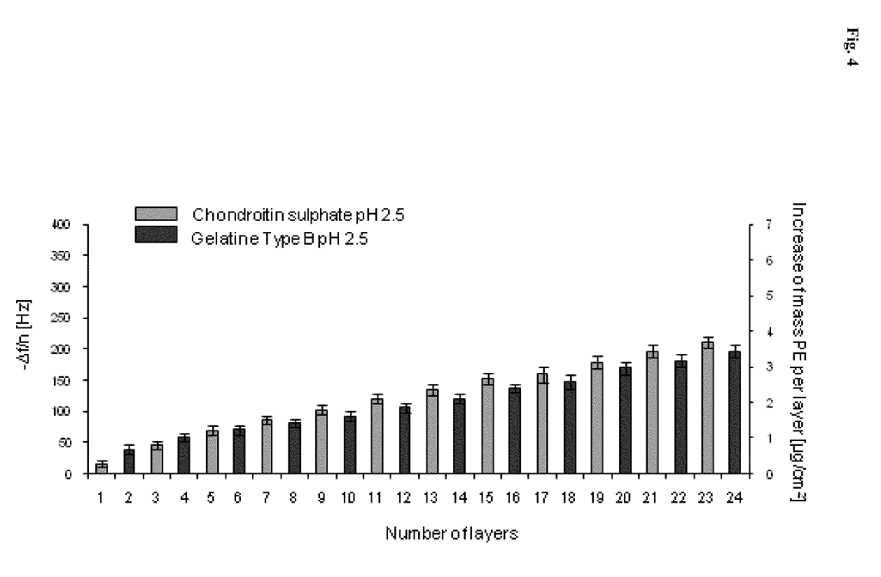

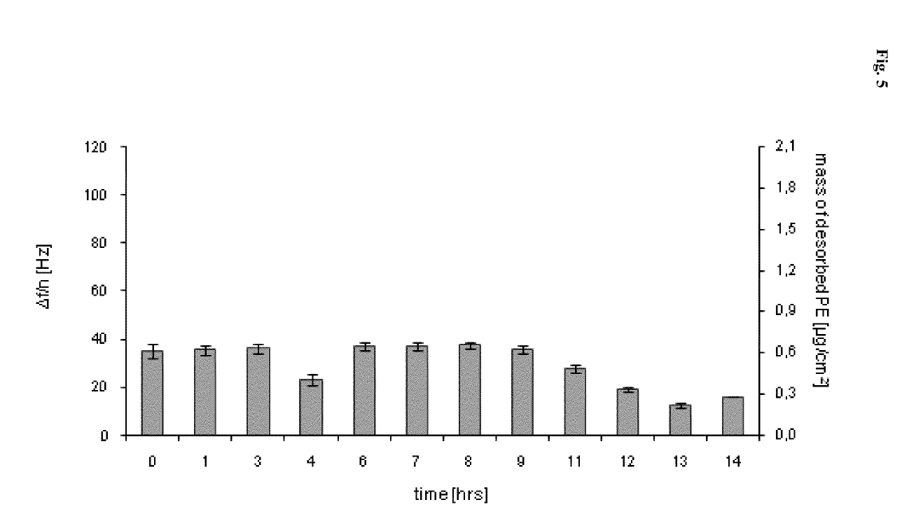

FIG. 4 and FIG. 5 show the progression of layer build-up and pH-dependent layer desorption, is respectively.

### Example 4

### Build-Up of Chondroitin Sulphate/Human Serum Albumin Polyelectrolyte Layers and Desorption vs. pH

### Materials: Layer Build-Up

PEI-[ChonS/HSA]12 at pH2.5
PE solution 1: Polyethylenimmine (PEI) (0.1 g/l) pH 2.5 containing 0.154 M NaCl
PE solution 2: Chondroitin sulphate (ChonS) (0.1 g/l) pH 2.5 containing 0.154 M NaCl
PE solution 3: Human Serum Albumin (HSA) (0.1 g/l) pH 2.5 containing 0.154 M NaCl
Washing water: MilliQ-Water adjusted to pH 2.5

### Materials: Desorption of Layers at pH 7.4

Phosphate buffer with Tween20 at pH 7.4

### Equipment:

Quartz Crystal Micro Balance (QCM-D)

### Pre-Treatment of Crystals:

to crystals to be coated were treated with polymer solution 1 (basic layer)

### LbL Coating:

after pre-treatment the crystals were coated with polymer solution 2 (anionic charge) at pH 2.5 for 2 min followed by a washing sequence with water at pH 2.5
subsequently the crystals were rinsed with polymer solution 3 (cationic charge) at pH 2.5 for 2 min followed by a washing sequence with water at pH 2.5
the washing and coating steps were repeated as required and according to the desired sequence of layers

### Sequence of Coating:

PEI-[ChonS/HSA]₁₂

### Desorption of PE Layers at pH 7.4:

rinse of crystals with PBS pH 7.4 solution for 1 min
residence time=29 min
total investigation time=16 hrs

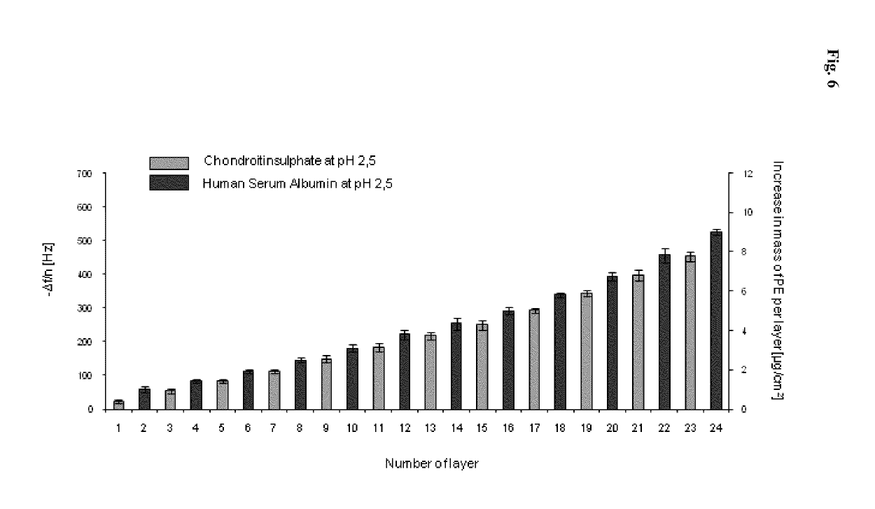

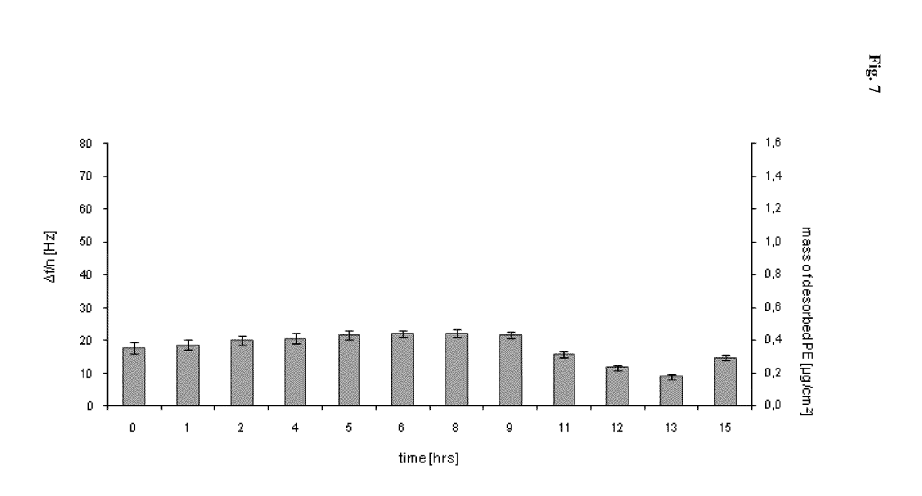

FIG. 6 and FIG. 7 show the progression of layer build-up and pH-dependent layer desorption, respectively.

### Example 5

### Coating of CaCO₃ Particles at pH 4 with Chondroitin Sulphate and a Model Protein at pH 3.5 and Release at pH 7.4

### Materials

CaCO₃ particles (Precarb 720)
Polymer solution 1: containing 4 mg/ml Polyethylenimmine (PEI) incl. 154 mM, pH 3.5 (cationic charged).
Polymer solution 2: containing 4 mg/ml Chondroitin sulphate (ChonS) incl. 154 mM NaCl, pH 3.5 (anionic charged).
Polymer solution 3: containing 0.8 mg/ml model protein (MP), pH 4 (cationic charged)
Washing: water pH 3.5 (adjusted with HCl/NaOH)

The model protein (MP) is a model for a therapeutic protein, i.e. a drug

### Pre-Treatment of Particles:

particles to be coated (PreCarb 720) were suspended in polymer solution 1 with a final particle concentration of 25 mg/ml in suspension

### LbL Coating:

the coating was performed in Eppendorf tubes of 2 ml volume
after pre-treatment the dispersion was centrifuged and the remaining particles were 3 times washed with washing water pH 3.5
subsequently polymer solution 2 was added and the particles were resuspended and coated for 10 min
subsequently the dispersion was centrifuged and the remaining particles were 3 times washed with washing water pH 3.5
subsequently polymer solution 3 was added and the particles were resuspended and coated for 10 min
the washing and coating steps were repeated as required and according to the desired sequence of layers; each coating step lasted 10 min;

### Sequence of Coating:

PEI-(ChonS-MP)₅

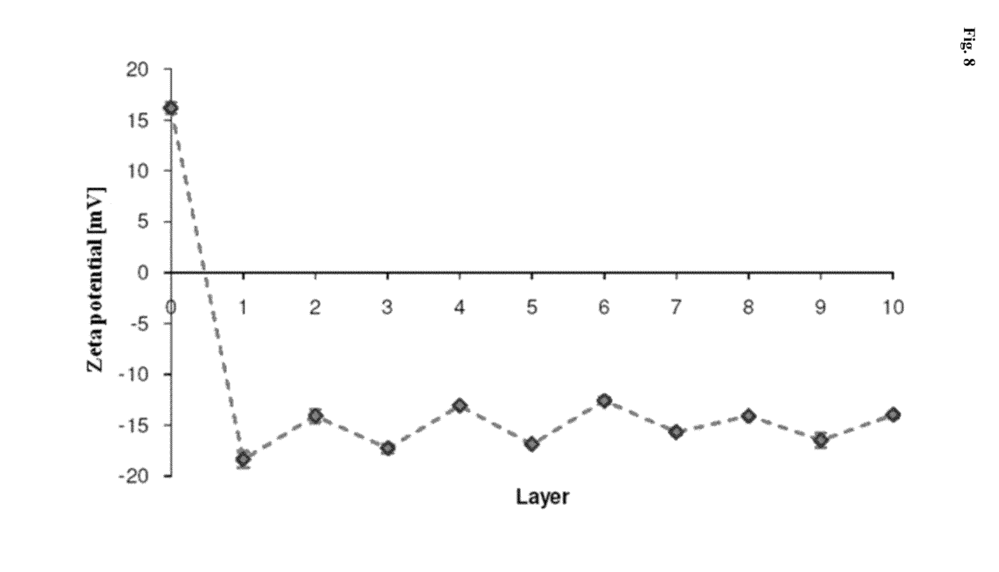

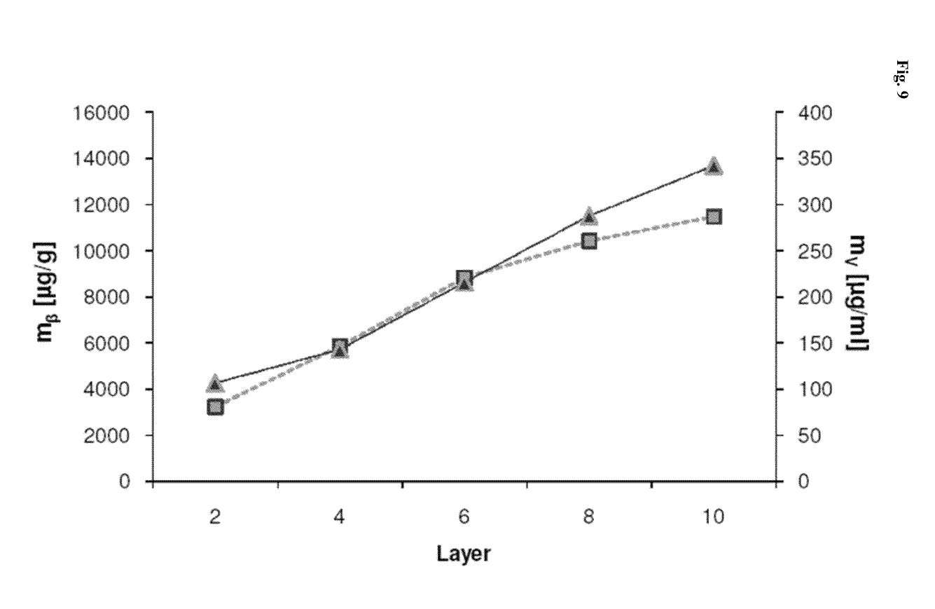

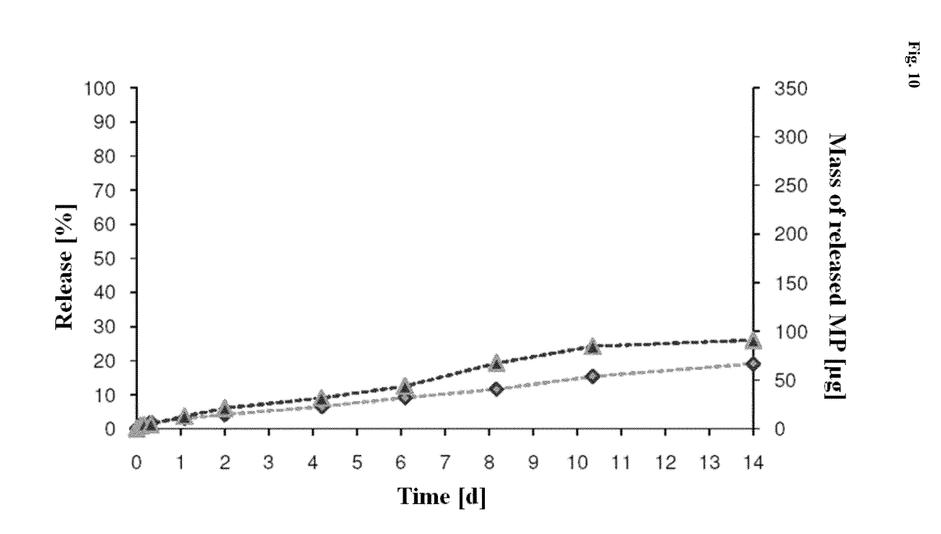

FIG. 8 illustrates the net charge of the coated particles in terms of zeta potential during coating. FIG. 9 illustrates the increase of the overall concentration of MP in the multilayer coating after 2, 4, 6, 8 and 10 layers of coating. FIG. 10 illustrates the release of the MP at physiological pH.

1. A method for coating a surface of a substrate with a drug for sustained release:
(i) providing a substrate with a surface to be coated,
(ii) depositing at least one bilayer on at least a portion of said surface,
wherein one layer of the bilayer comprises a polyelectrolyte and the other layer comprises a pharmaceutically active ingredient, and
wherein the two layers of the bilayer are oppositely charged under conditions of the deposition and at least one layer of the bilayer has a substantially different net charge under physiological conditions.

2. The method according to claim 1, wherein said two layers of the bilayer have the same net charge under physiological conditions.

3. The method according to claim 1, wherein depositing said at least one bilayer on the surface takes place at a pH<6 or a pH>8.

4. The method according to claim 1, wherein said pharmaceutically active ingredient is an amphoteric substance which comprises an isoelectric point IEP>9.4 or IEP<5.4.

5. The method according to claim 1, wherein said polyelectrolyte is an amphoteric substance which comprises an isoelectric point IEP>9.4 or IEP<5.4.

6. The method according to claim 1, wherein a plurality of bilayers is deposited on at least the portion of a surface to form a multilayer of layers with alternating charges.

7. The method according to claim 1, wherein the substrate is a particle and/or a porous particle and/or an implantable and/or insertable medical device.

8. The method according to claim 1, wherein the polyelectrolyte is selected from the group consisting of synthetic polymers, biopolymers such as polypeptides, proteins, polysaccharides, oligosaccharides, nucleic acids and derivates of biopolymers optionally comprising a chemically modified biological polymer.

9. The method according to claim 1, wherein the pharmaceutically active ingredient comprises a water soluble drug according to groups I or III of Biopharmaceutics Classification System (BCS) (FDA).

10. The method according to claim 1, wherein the layer comprising the pharmaceutically active ingredient further comprises a polyelectrolyte, an organic and/or inorganic salt, and/or organic and/or inorganic particle with low water solubility.

11. A substrate comprising
(i) a surface, and
(ii) a multilayer coating at least on a portion of said surface,
wherein the multilayer comprises at least one bilayer having two layers of opposite charge at deposition and storage conditions, and
wherein one layer of the bilayer comprises a polyelectrolyte and the other layer comprises
a pharmaceutically active ingredient, and
wherein one layer of the bilayer substantially changes net charge when subjected to physiological conditions.

12. The substrate according to claim 11, wherein said substrate is a microscopic particle and/or bead.

13. The substrate comprising a particle and/or porous particle according to claim 12, capable of being used in treatment of a disease optionally that is at least one selected from the group consisting of tumours, vascular diseases or circulatory disturbances, ophthalmic diseases, gynecological diseases and conditions, articular and bone diseases, for facilitating wound healing and/or the healing of bone fractures.

14. The substrate according to claim 11, wherein said substrate is an implantable and/or insertable medical device.

15. The substrate comprising an implantable and/or insertable medical device according to claim 14, capable of being used in treatment of a tumour, for creating open passage in the body, for treatment of vascular disease and/or circulatory disturbance, for facilitating wound healing and/or healing of bone fracture, for treatment of ophthalmic disease, in surgery, for treatment of gynecological disease and/or condition, for treatment of articular and/or bone disease, for treatment of stenosis and/or in prophylaxis of restenosis.
